(19) United States
(12) Patent Application Publication
(10) Pub. No.: US 2023/0368906 A1
(43) Pub. Date: Nov. 16, 2023

(54) METHOD AND SYSTEM FOR GENERATING DATA ANALYSIS REPORT OF MULTI-PARAMETER MONITORING DEVICE

(71) Applicant: Shanghai SVM Medical Technology Co., Ltd., Shanghai (CN)

(72) Inventors: Xiaobo HUANG, Shanghai (CN); Yun LONG, Shanghai (CN); Yundai CHEN, Shanghai (CN); Longxiang SU, Shanghai (CN); Yunping LAN, Shanghai (CN); Song ZHANG, Shabghai (CN); Qian ZHAI, Shanghai (CN); Baoshi HAN, Shanghai (CN); Hongli HE, Shanghai (CN); Haiquan CAO, Shanghai (CN); Xinkang WANG, Shanghai (CN); Xiangde ZHENG, Shanghai (CN); Yi WU, Shanghai (CN); Sian SUN, Shanghai (CN); Dong WANG, Shanghai (CN); Zujun TANG, Shanghai (CN); Bin ZHU, Shanghai (CN); Haoyu YANG, Shabghai (CN); Jian SUN, Shanghai (CN); Jinjing ZHANG, Shanghai (CN)

(73) Assignee: Shanghai SVM Medical Technology Co., Ltd., Shanghai (CN)

(21) Appl. No.: 17/768,477

(22) PCT Filed: Dec. 30, 2020
(86) PCT No.: PCT/CN2020/141363

§ 371 Date: Apr. 13, 2022

(30) Foreign Application Priority Data
Feb. 18, 2020 (CN) 202010100416.6

(51) Int. Cl. G16H 40/63

(52) U.S. Cl. CPC G16H 40/63

(57) ABSTRACT

Disclosed are a method and system for generating a data analysis report of a multi-parameter monitoring device. The method includes: receiving, by an application server, multi-parameter data transmitted in real time by the multi-parameter monitoring device, and storing the received multi-parameter data in a database server; the application server receiving a report generation instruction transmitted by a client, the instruction including a client identifier used for indicating a permission of the client; if the permission is a first mode permission, transmitting the multi-parameter data to the client, to allow the client to process the multi-parameter data, so as to generate a multi-parameter analysis report and a report attachment; and if the permission is a second mode permission, the application server processing the multi-parameter data to generate the multi-parameter analysis report and the report attachment, and transmitting the multi-parameter analysis report and the report attachment to the client.

### TECHNICAL FIELD

The present disclosure relates to the field of medical device data processing technologies, and in particular, to a method and system for generating a data analysis report of a multi-parameter monitoring device.

### BACKGROUND

As an important device in the medical field, a multi-parameter monitoring device is used to monitor changes in physiological and pathological data of critically ill patients. According to data change trends and evolution processes provided by the device, a doctor evaluates changes and severity of a patient's condition, so as to make a clinical decision and formulate a medical treatment plan. For example, a patient's heart rate and respiratory rate are on an upward trend, and a body temperature is on a downward/upward trend, based on which a doctor can determine that the patient's body is fighting an infection, thereby providing further diagnosis and treatment to prevent disease progression.

Over the past decade, the global multi-parameter monitoring device technology has made great progress, which possesses the ability to generate hundreds of MBytes of physiological and pathological data within 24 hours. However, clinical application modes of the multi-parameter data have not been advanced accordingly. The existing multi-parameter monitoring devices from various factories lack the technology to generate data analysis reports. Thus, when an alarm is issued, it is required that a medical staff retrieves data from a bedside multi-parameter monitoring device or a multi-parameter monitoring central workstation, and performs manual analysis on a device screen. In addition, it takes a lot of time to view all of the patient's data. In most cases, the medical staff only retrieves segment data for viewing. Recently, multi-parameter monitoring devices from some factories are equipped with a printing function, but it still takes a lot of time for the medical staff to view and select data on the screen, and the printout is only a record of partial data. Obviously, these two application modes of the multi-parameter data do not allow the medical staff to comprehensively understand change trends and processes of the patient's physiological and pathological data, thus being not conducive to diagnosis and treatment. Moreover, since the medical staff is generally not skilled at multi-parameter data analysis, there may be a risk of medical errors caused by missed diagnosis and misdiagnosis. At present, about tens of millions of multi-parameter monitoring devices are in use in hospitals around the world, and thus there is an urgent need to solve the above problem.

### SUMMARY

In view of the above analysis, the present disclosure provides a method and system for generating a data analysis report of a multi-parameter monitoring device, aiming to solve the problem that the existing multi-parameter monitoring device technology lacks the ability to generate data analysis reports.

The objective of the present application is achieved by the following technical solutions.

An aspect provides a method for generating a data analysis report of a multi-parameter monitoring device, including:

receiving, by an application server, multi-parameter data transmitted in real time by the multi-parameter monitoring device, and storing the received multi-parameter data in a database server;

receiving, by the application server, a report generation instruction transmitted by a client, the instruction including a client identifier that is configured for indicating a permission of the client; and

in a case that the permission of the client is a first mode permission (active mode permission), transmitting, by the application server, the multi-parameter data to the client, to allow the client to process the multi-parameter data, so as to generate a multi-parameter analysis report and a report attachment; and in a case that the permission of the client is a second mode permission (passive mode permission), processing, by the application server, the multi-parameter data to generate the multi-parameter analysis report and the report attachment, and transmitting the multi-parameter analysis report and the report attachment to the client.

On the basis of the foregoing solution, following improvements have been made.

Further, after the application server receives the multi-parameter data transmitted in real time by the multi-parameter monitoring device, the method also includes: configuring a unique identifier for the multi-parameter data;

storing the configured unique identifier in a first storage area of the database server, and establishing a unique identifier index based on the unique identifier;

storing the received multi-parameter data in a second storage area of the database server, and marking the multi-parameter data according to the unique identifier of the multi-parameter data; and

storing the generated multi-parameter analysis report and the report attachment in a third storage area of the database server, and marking the generated multi-parameter analysis report and the report attachment according to the unique identifier of the multi-parameter data.

The unique identifier index established by the first storage area has a mutual mapping relationship with the second storage area and the third storage area, respectively, to provide unified management of information stored in the first storage area, the second storage area, and the third storage area that is associated by the unique identifier.

Further, after the application server receives the multi-parameter data transmitted in real time by the multi-parameter monitoring device, the method also includes:

counting, by the application server, a time length of the received multi-parameter data;

in a case that the time length of the received multi-parameter data reaches a predetermined time length, transmitting the unique identifier index of the multi-parameter data of the predetermined time length to the client;

in a case that the time length of the multi-parameter data is less than the predetermined time length and no longer increases, transmitting the unique identifier index of the multi-parameter data whose time length is less than the predetermined time length and no longer increases to the client; and

transmitting, by the client, the report generation instruction to the application server according to the received unique identifier index.

Further, the report generation instruction also includes a data time period parameter for report generation;

the application server determines to-be-processed multi-parameter data of a corresponding time period length based on the data time period parameter for report generation, and generate the multi-parameter analysis report and the report attachment based on the determined multi-parameter data of the corresponding time period length.

Further, the multi-parameter data includes patient information, waveform data, and numerical data.

Further, the generation of the multi-parameter analysis report and the report attachment includes:

processing the multi-parameter data, to obtain measurement statistical values, analysis statistical charts, time series identifiers, and event identifiers, the measurement statistical values including an average value, a maximum value, a minimum value, and a variability analysis value;

importing the measurement statistical values, the time series identifiers, the event identifiers, and patient basic information obtained by mapping the unique identifier index into a multi-parameter analysis report framework, to generate the multi-parameter analysis report; and

importing the measurement statistical values, the analysis statistical charts, the time series identifiers, the event identifiers, the multi-parameter waveform data, and the numerical data into a multi-parameter analysis report attachment framework, to generate the multi-parameter analysis report attachment.

The multi-parameter analysis report is configured for displaying a change trend of each parameter and a data analysis conclusion, and the report attachment is configured for displaying an analysis report of each parameter including the waveform data and the numerical data.

Further, the multi-parameter analysis report framework includes at least a patient basic information area; and

the multi-parameter analysis report framework also includes:

one or more of following parameter data trend analysis areas: a heart rate data trend analysis area, a blood pressure data trend analysis area, a respiratory rate data trend analysis area, a blood oxygen saturation level data trend analysis area, a body temperature data trend analysis area, and a designated data trend analysis area; and

a data analysis conclusion area.

Further, the report generation instruction also includes a setting parameter of the designated data trend analysis area, according to which the application server sets content of the designated data trend analysis area; and

the report generation instruction also includes a setting parameter of the data analysis conclusion area, according to which the application server sets content of the data analysis conclusion area.

Further, the parameter data trend analysis area has an ordinate set as a numerical scale and an abscissa set as a time scale, configured for displaying a state in a unit time and a change process of each parameter, and includes at least the measurement statistical values, the time series identifiers, and the event identifiers of each parameter.

Further, the designated data trend analysis area is configured for displaying a trend analysis graph of parameter data that is preset and needs intensive monitoring for patients with different diseases.

The parameter data that needs intensive monitoring includes one or more of following parameters: a perfusion index, a partial pressure of end-tidal carbon dioxide, an airway flow/pressure, a central venous pressure, a pulse pressure, a stroke volume, an intracranial pressure, and a bispectral index.

Further, each of the parameter data trend analysis areas in the multi-parameter analysis report is associated with each parameter analysis page in the report attachment. In response to clicking on a certain data trend analysis area on a screen displaying the multi-parameter analysis report, the multi-parameter analysis report automatically jumps to a corresponding parameter analysis page, to allow a user to view page by page the measurement statistical values, the analysis statistical charts, the waveform data, the numerical data, and marked event data, and perform a zoom in operation, a zoom out operation, or a scrolling operation on the screen for viewing, and select a range of data for viewing.

Further, the client with the first mode permission and the client with the second mode permission are also configured to transmit the multi-parameter analysis report, the report attachment, and the unique identifier index to an associated medical staff terminal, after the multi-parameter analysis report and the report attachment are generated.

Further, the method also includes:

receiving, by the application server, a report acquisition instruction transmitted by the medical staff terminal, the report acquisition instruction including a unique identifier index of a patient or a statistical parameter;

in a case that the unique identifier index is included, retrieving, by the application server, a multi-parameter analysis report and a report attachment according to the unique identifier index, and transmitting the multi-parameter analysis report and the report attachment to the medical staff terminal; and

in a case that the statistical parameter is included, retrieving, by the application server, a multi-parameter analysis report and a report attachment that satisfy the statistical parameter, and transmitting the multi-parameter analysis report and the report attachment to the medical staff terminal.

Further, the method also includes:

receiving, by the application server, a report archiving instruction transmitted by a client or a medical staff terminal with a report archiving permission, the report archiving instruction including the unique identifier index; and retrieving, by the application server, a multi-parameter analysis report and a report attachment according to the unique identifier index, and transmitting the multi-parameter analysis report and the report attachment to a hospital information management system, an electronic medical record system, or a critical clinical information system, to store the multi-parameter analysis report and the report attachment in a patient electronic medical record.

Further, the application server receiving the report generation instruction transmitted by the client also includes:

receiving, by the application server, the report generation instruction that is transmitted by a client browser and successfully authenticated by a world wide web (WEB) server, the report generation instruction including a client browser identifier, and the permission of the client indicated by the client browser identifier being the second mode permission.

Further, the application server receiving the multi-parameter data transmitted in real time by the multi-parameter monitoring device includes:

connecting, by a mobile storage apparatus, to an interface of the multi-parameter monitoring device, to download the multi-parameter data; and

connecting, by the client, to an interface of the mobile storage apparatus, to upload read multi-parameter data to the application server.

Another aspect provides a system for generating a data analysis report of a multi-parameter monitoring device, including:

one or more clients, configured to transmit a report generation instruction to an application server, the report generation instruction including a client identifier that is configured for indicating a permission of the client;

the application server, configured to receive multi-parameter data transmitted in real time by the multi-parameter monitoring device; and further configured to receive the report generation instruction transmitted by the client, in a case that the permission of the client is a first mode permission, transmit the multi-parameter data to the client, to allow the client to process the multi-parameter data, so as to generate a multi-parameter analysis report and a report attachment, and in a case that the permission of the client is a second mode permission, process the multi-parameter data to generate the multi-parameter analysis report and the report attachment, and transmit the multi-parameter analysis report and the report attachment to the client; and

a database server, configured to store the multi-parameter data received by the application server.

On the basis of the foregoing solution, following improvements have been made.

The application server is also configured to configure a unique identifier for the multi-parameter data after receiving the multi-parameter data transmitted in real time by the multi-parameter monitoring device.

The database server includes a first storage area, a second storage area, and a third storage area.

The first storage area of the database server is configured to store the configured unique identifier, and establish a unique identifier index based on the unique identifier.

The second storage area of the database server is configured to store the multi-parameter data received by the application server, and mark the multi-parameter data according to the unique identifier of the multi-parameter data.

The third storage area of the database server is configured to store the generated multi-parameter analysis report and the report attachment, and mark the generated multi-parameter analysis report and the report attachment according to the unique identifier of the multi-parameter data.

The unique identifier index established by the first storage area has a mutual mapping relationship with the second storage area and the third storage area, respectively, to provide unified management of information stored in the first storage area, the second storage area, and the third storage area that is associated by the unique identifier.

Further, after receiving the multi-parameter data transmitted in real time by the multi-parameter monitoring device, the application server is configured to perform the following operations:

counting a time length of the received multi-parameter data;

in a case that the time length of the received multi-parameter data reaches a predetermined time length, transmitting the unique identifier index of the multi-parameter data of the predetermined time length to the client; and

in a case that the time length of the multi-parameter data is less than the predetermined time length and no longer increases, transmitting the unique identifier index of the multi-parameter data whose time length is less than the predetermined time length and no longer increases to the client.

The client is also configured to transmit the report generation instruction to the application server according to the received unique identifier index.

Further, the report generation instruction also includes a data time period parameter for report generation.

The application server is also configured to determine to-be-processed multi-parameter data of a corresponding time period length based on the data time period parameter for report generation, and generate the multi-parameter analysis report and the report attachment based on the determined multi-parameter data of the corresponding time period length.

Further, the multi-parameter data includes patient information, waveform data, and numerical data.

Further, the generation of the multi-parameter analysis report and the report attachment includes:

processing the multi-parameter data, to obtain measurement statistical values, analysis statistical charts, time series identifiers, and event identifiers, the measurement statistical values including an average value, a maximum value, a minimum value, and a variability analysis value;

importing the measurement statistical values, the time series identifiers, the event identifiers, and patient basic information obtained by mapping the unique identifier index into a multi-parameter analysis report framework, to generate the multi-parameter analysis report; and

importing the measurement statistical values, the analysis statistical charts, the time series identifiers, the event identifiers, the multi-parameter waveform data, and the numerical data into a multi-parameter analysis report attachment framework, to generate the multi-parameter analysis report attachment.

The multi-parameter analysis report is configured for displaying a change trend of each parameter and a data analysis conclusion, and the report attachment is configured for displaying an analysis report of each parameter including the waveform data and the numerical data.

Further, the multi-parameter analysis report framework includes at least a patient basic information area; and

the multi-parameter analysis report framework further includes:

one or more of following parameter data trend analysis areas: a heart rate data trend analysis area, a blood pressure data trend analysis area, a respiratory rate data trend analysis area, a blood oxygen saturation level data trend analysis area, a body temperature data trend analysis area, and a designated data trend analysis area; and

a data analysis conclusion area.

Further, the report generation instruction also includes a setting parameter of the designated data trend analysis area, according to which the application server sets content of the designated data trend analysis area; and

the report generation instruction also includes a setting parameter of the data analysis conclusion area, according to which the application server sets content of the data analysis conclusion area.

Further, the parameter data trend analysis area has an ordinate set as a numerical scale and an abscissa set as a time scale, configured for displaying a state in a unit time and a change process of each parameter, and includes at least the measurement statistical values, the time series identifiers, and the event identifiers of each parameter.

Further, the designated data trend analysis area is configured for displaying a trend analysis graph of parameter data that is preset and needs intensive monitoring for patients with different diseases.

The parameter data that needs intensive monitoring includes one or more of following parameters: a perfusion index, a partial pressure of end-tidal carbon dioxide, an airway flow/pressure, a central venous pressure, a pulse pressure, a stroke volume, an intracranial pressure, and a bispectral index.

Further, each of the parameter data trend analysis areas in the multi-parameter analysis report is associated with each parameter analysis page in the report attachment. In response to clicking on a certain data trend analysis area on a screen displaying the multi-parameter analysis report, the multi-parameter analysis report automatically jumps to a corresponding parameter analysis page, to allow a user to view page by page the measurement statistical values, the analysis statistical charts, the waveform data, the numerical data, and marked event data, and perform a zoom in operation, a zoom out operation, or a scrolling operation on the screen for viewing, and select a range of data for viewing.

Further, the client with the first mode permission and the client with the second mode permission are also configured to transmit the multi-parameter analysis report, the report attachment, and the unique identifier index to an associated medical staff terminal, after the multi-parameter analysis report and the report attachment are generated.

Further, the system also includes a medical staff terminal, configured to transmit a report acquisition instruction to the application server, the report acquisition instruction including a unique identifier index of a patient or a statistical parameter.

The application server is also configured to receive the report acquisition instruction transmitted by the medical staff terminal, in a case that the unique identifier index is included, retrieve a multi-parameter analysis report and a report attachment according to the unique identifier index, and transmit the multi-parameter analysis report and the report attachment to the medical staff terminal; and

in a case that the statistical parameter is included, retrieve a multi-parameter analysis report and a report attachment that satisfy the statistical parameter, and transmit the multi-parameter analysis report and the report attachment to the medical staff terminal.

Further, the application server is also configured to receive a report archiving instruction transmitted by a client or a medical staff terminal with a report archiving permission, the report archiving instruction including the unique identifier index, retrieve a multi-parameter analysis report and a report attachment according to the unique identifier index, and transmit the multi-parameter analysis report and the report attachment to a hospital information management system, an electronic medical record system, or a critical clinical information system, to store the multi-parameter analysis report and the report attachment in a patient electronic medical record.

Further, the system also includes a WEB server.

The application server is configured to receive the report generation instruction that is transmitted by a client browser and successfully authenticated by the WEB server, the report generation instruction including a client browser identifier, and the permission of the client indicated by the client browser identifier being the second mode permission.

Further, the system also includes a mobile storage apparatus.

The application server is configured to receive the multi-parameter data transmitted by the multi-parameter monitoring device, including:

the mobile storage apparatus connects to an interface of the multi-parameter monitoring device, to download the multi-parameter data; and

the client connects to an interface of the mobile storage apparatus, to upload read multi-parameter data to the application server.

The present disclosure has the following beneficial effects.

According to the method and system for generating a data analysis report of a multi-parameter monitoring device according to the present disclosure:

1. The method and system can make full use of the multi-parameter data outputted by the multi-parameter monitoring device, to calculate to generate the multi-parameter analysis report and the report attachment. By viewing the multi-parameter analysis report and the report attachment, the medical staff can quickly and comprehensively understand change trends and processes of patient's physiological and pathological data, so as to formulate a diagnosis and treatment plan and a nursing care plan, which improves quality and efficiency of medical treatment. The present disclosure overcomes the deficiency that the existing technology lacks the ability to generate the multi-parameter analysis report, thus having a wide range of applicability.

2. The present disclosure has good distribution and scalability, which is able to manage large-scale multi-parameter monitoring devices, client devices, and medical staff terminals. A hospital can centrally process multi-parameter data services with a huge amount of data scattered in various departments, and permanently save the multi-parameter data and the report files, which improves work efficiency and reduces service costs. The present disclosure may also be applied to provide data services for multi-parameter monitoring devices in lower-level hospitals, which overcomes the shortcomings of the existing technology, thus having a wide range of applicability.

3. The multi-parameter analysis report and the report attachment generated according to the present disclosure provide the change trends, the event statuses and the time points of each parameter data during a patient's medical treatment process, as well as the data analysis conclusion, so that the medical staff can understand the change trends and processes of the patient's physiological and pathological data and the disease severity, so as to provide a precise intervention, which improves quality and efficiency of medical treatment, thereby alleviating work burden on the medical staff. The present disclosure overcomes the deficiencies of the existing technology in the clinical application, thus having a wide range of applicability.

The above technical solutions in the present disclosure can also be combined with each other to realize more preferred combination solutions thereof Other features and advantages of the present application will be described in the following specification, and some of these will become apparent from the specification or be understood by implementing the present application. The objectives and other advantages of the present application can be implemented or obtained by structures specifically indicated in the written specification, claims, and accompanying drawings.

### BRIEF DESCRIPTION OF THE DRAWINGS

The accompanying drawings are provided merely for illustrating specific examples and are not considered as limiting the present application. Throughout the accompanying drawings, the same reference numerals represent the same components.

FIG. 1 is a flowchart of a method for generating a data analysis report of a multi-parameter monitoring device according to embodiment 1 of the present disclosure.

FIG. 2 is a framework diagram of a multi-parameter data analysis report generated according to embodiment 1 of the present disclosure.

FIG. 3 is a schematic diagram of viewing an attachment of a multi-parameter analysis report generated according to embodiment 1 of the present disclosure.

FIG. 4 is a framework diagram of a system for generating a data analysis report of a multi-parameter monitoring device in a client/server (C/S) architecture according to embodiment 3 of the present disclosure.

FIG. 5 is a framework diagram of a system for generating a data analysis report of a multi-parameter monitoring device in a browser/server (B/S) architecture according to embodiment 3 of the present disclosure.

FIG. 6 is a framework diagram of a system for generating a data analysis report of a multi-parameter monitoring device in a local client architecture according to embodiment 3 of the present disclosure.

### DETAILED DESCRIPTION OF THE EMBODIMENTS

Preferred examples of the present application will be described in detail below with reference to the accompanying drawings. The accompanying drawings constitute a part of the present application, and are used together with the examples of the present application for explaining principles of the present application rather than for limiting a scope of the present application.

### Embodiment 1

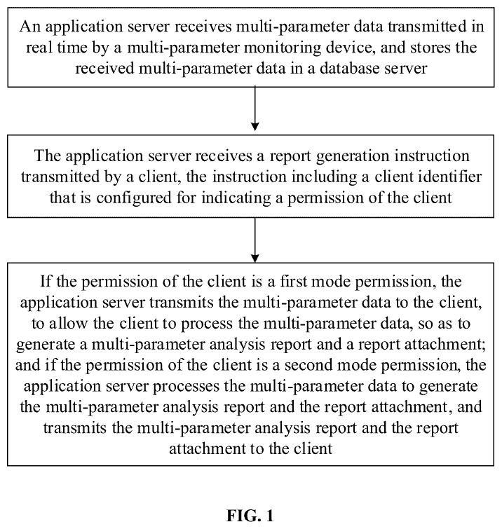

A specific embodiment of the present disclosure provides a method for generating a data analysis report of a multi-parameter monitoring device, a flowchart of which is shown in FIG. 1. The method includes the following operations:

Step S1. An application server receives multi-parameter data transmitted in real time by the multi-parameter monitoring device, and stores the received multi-parameter data in a database server.

Step S2. The application server receives a report generation instruction transmitted by a client. The instruction includes a client identifier, which is configured for indicating a permission of the client.

Step S3. If the permission of the client is a first mode permission, the application server transmits the multi-parameter data to the client, to allow the client to process the multi-parameter data, so as to generate a multi-parameter analysis report and a report attachment. If the permission of the client is a second mode permission, the application server processes the multi-parameter data to generate the multi-parameter analysis report and the report attachment, and transmits the multi-parameter analysis report and the report attachment to the client.

After the multi-parameter analysis report and the report attachment are generated, the method may also include the following operation:

Step S4. After the multi-parameter analysis report and the report attachment are generated, the client with the first mode permission and the client with the second mode permission transmit the multi-parameter analysis report and the report attachment to an associated medical staff terminal.

Compared with the related art, the method in the present disclosure can make full use of the multi-parameter data outputted by the multi-parameter monitoring device, to generate the multi-parameter analysis report and the report attachment. By viewing the multi-parameter analysis report and the report attachment, the medical staff can quickly and comprehensively understand change trends and processes of patient's physiological and pathological data, so as to formulate a diagnosis and treatment plan and a nursing care plan. In addition, the present disclosure has good distribution and scalability, which has the ability to manage large-scale multi-parameter monitoring devices, client devices, and medical staff terminals. The hospital can centrally process multi-parameter data services with a huge amount of data scattered in various departments, and permanently save the multi-parameter data and the report files, thereby reducing service costs. The medical staff terminal may view, retrieve, and query report files at any time, which improves quality and efficiency of medical treatment. Moreover, the present disclosure may receive data from a multi-parameter device at a low-level hospital and then provide the multi-parameter analysis report and the report attachment to the low-level hospital, which overcomes the deficiencies of the existing technology in the clinical application, thus having a wide range of applicability.

The method provided by the present disclosure is adapted for an actual situation of a medical process. Specifically, in the medical field, since the data analysis is highly professional and the medical staff is generally not skilled at the data analysis, there may exist a risk of medical errors caused by missed diagnosis and misdiagnosis. Thus, the client is provided in the method, configured to cooperate with the application server for generating the multi-parameter analysis report and the report attachment.

Optionally, in the method, the permission of the client may be determined according to different application scenarios and requirements of hospitals, which is not limited herein. For example, in some embodiments, the hospital may only limit the functions of the application server.

In this embodiment, the permission of the client refers to whether the client itself has a report generation function, which may be indicated by a client device identifier. For the purpose of system management and system security, all client device identifiers indicating the report generation function need to be stored in the application server. In this case, the client with the first mode permission can generate the multi-parameter analysis report and the report attachment relying on its excellent storage capacity and computing power; that is, the client with the first mode permission itself has the report generation function. For example, the client device identifier may be a desktop computer. The client with the second mode permission does not have the report generation function due to its weak storage capacity and computing power, and in this case, the application server needs to be used to generate the multi-parameter analysis report and the report attachment. For example, the client identifier may be a mobile device. Accordingly, Step 3 includes the following operations:

Step S31. The application server determines whether the client device identifier has been stored. If the client device identifier has been stored, step S32 is performed; and if the client device identifier has not been stored, the application server transmits feedback information of “not having the report generation function” to the client.

Step S32. If the client device identifier is the desktop computer, the application server transmits the multi-parameter data to the client, to allow the client to process the multi-parameter data, so as to generate the multi-parameter analysis report and the report attachment. If the client device identifier is the mobile device, the application server processes the multi-parameter data to generate the multi-parameter analysis report and the report attachment.

It can be envisaged that as technologies advance, the mobile device will be developed to have more powerful storage capacity and computing power. At that time, the application server may redefine the client device identifier, to determine the process of generating the multi-parameter analysis report and the report attachment is carried out in the client or in the application server.

In other embodiments, the hospital may limit the functions of the client and the application server at the same time. The permission of the client may be specifically set according to an actual situation, which is more flexible. It is necessary to limit the application server correspondingly, to ensure that the application server effectively identifies the permission of the client, and determines the process of generating the multi-parameter analysis report and the report attachment is carried out in the client or in the application server according to the permission of the client.

Optionally, in the above step S1, after the application server receives the multi-parameter data transmitted in real time by the multi-parameter monitoring device, the method also includes: configuring a unique identifier for the multi-parameter data; storing the configured unique identifier in a first storage area of the database server, and establishing a unique identifier index based on the unique identifier. For example, the unique identifier may be a combination of various pieces of clinical information and monitoring information, such as a device number, a hospitalization number, a data sampling time, and etc. The unique identifier index is a storage structure that points to data and is established in the first storage area of the database server based on the unique identifier, which is configured for data retrieval, query, statistical, and management. The application server also stores the received multi-parameter data in a second storage area of the database server, and marks the multi-parameter data according to the unique identifier of the multi-parameter data. In addition, the application server also stores the generated multi-parameter analysis report and report attachment in a third storage area of the database server, and marks the generated multi-parameter analysis report and report attachment according to the unique identifier of the multi-parameter data. The unique identifier index established by the first storage area has a mutual mapping relationship with the second storage area and the third storage area, respectively, to realize unified management of information stored in the first storage area, the second storage area, and the third storage area that is associated by the unique identifier. In this way, the unified management of the multi-parameter data and the report files can be realized, which facilitates retrieval of the multi-parameter data and the report files, data service scheduling, and statistical analysis.

Optionally, in the above step S1, after the application server receives the multi-parameter data transmitted in real time by the multi-parameter monitoring device, the method also includes: the application server counting a time length of the received multi-parameter data; if the time length of the received multi-parameter data reaches a predetermined time length, transmitting the unique identifier index of the multi-parameter data of the predetermined time length to the client; and if the time length of the multi-parameter data is less than the predetermined time length and no longer increases, transmitting the unique identifier index of the multi-parameter data whose time length is less than the predetermined time length and no longer increases to the client. The client transmits the report generation instruction to the application server according to the received unique identifier index. In this way, the multi-parameter analysis report and the report attachment can be generated regularly, thereby better satisfying user requirements. The predetermined time length may be set according to user requirements or patient diagnosis and treatment requirements, such as, 24 hours.

Optionally, in an actual application, there may be a requirement to temporarily generate an analysis report of a specific time period. In this case, a staff may set a data time period parameter by the client, and load the data time period parameter in the report generation instruction that is to be transmitted to the application server. Based on the data time period parameter for report generation, the application server determines to-be-processed multi-parameter data of a corresponding time period length, and generates the multi-parameter analysis report and the report attachment according to the determined multi-parameter data of the corresponding time period length.

Optionally, in the above step S1, the application server may receive the multi-parameter data transmitted in real time by the multi-parameter monitoring device in a variety of manners, such as, in a wireless/wired network transmission manner. In a case that network transmission is not allowed in certain environments, the multi-parameter data may be transmitted as follows to ensure a smooth process: a mobile storage apparatus is connected to an interface of the multi-parameter monitoring device, to download the multi-parameter data; and the client is connected to an interface of the mobile storage apparatus, to upload the read multi-parameter data to the application server.

Optionally, the multi-parameter monitoring device includes at least a multi-parameter monitor and a multi-parameter monitoring central workstation. The multi-parameter data includes patient information, waveform data, and numerical data. The client and the application server both perform the following process to generate the multi-parameter analysis report and the report attachment.

The multi-parameter data is processed, to obtain measurement statistical values, time series identifiers, event identifiers, and analysis statistical charts. The analysis statistical charts may be selected from one or more of the following: a bar graph, a pie chart, a line graph, a histogram, a scatter plot, and a variability analysis chart. The measurement statistical values include: an average value, a maximum value, a minimum value, and a variability analysis value. The measurement statistical values, the time series identifiers, the event identifiers, and patient basic information obtained by mapping the unique identifier index are imported into a multi-parameter analysis report framework, to generate the multi-parameter analysis report. The measurement statistical values, the analysis statistical charts, the time series identifiers, the event identifiers, the multi-parameter waveform data, and the numerical data are imported into a multi-parameter analysis report attachment framework, to generate the multi-parameter analysis report attachment.

The multi-parameter analysis report is configured to display a change trend of each parameter and a data analysis conclusion. The report attachment is configured to display an analysis report of each parameter including the multi-parameter waveform data and the numerical data.

Optionally, the multi-parameter analysis report framework includes at least a patient basic information area; and in addition, includes one or more of the following parameter data trend analysis areas: a heart rate data trend analysis area, a blood pressure data trend analysis area, a respiratory rate data trend analysis area, a blood oxygen saturation level data trend analysis area, a body temperature data trend analysis area, and a designated data trend analysis area; and a data analysis conclusion area.

Optionally, when the multi-parameter analysis report framework includes the designated data trend analysis area, the report generation instruction also includes a setting parameter of the designated data trend analysis area, according to which the application server sets content of the designated data trend analysis area. It should be noted that the designated data trend analysis area is configured to display a trend analysis graph of parameter data that is preset and needs intensive monitoring for patients with different diseases. The parameter data needing the intensive monitoring includes one or more of the following: a perfusion index, a partial pressure of end-tidal carbon dioxide, an airway flow/pressure, a central venous pressure, a pulse pressure, a stroke volume, an intracranial pressure, and a bispectral index. In addition, the report generation instruction also includes a setting parameter of the data analysis conclusion area, according to which the application server sets content of the data analysis conclusion area.

Optionally, the parameter data trend analysis area has an ordinate set as a numerical scale and an abscissa set as a time scale, configured for displaying a state in a unit time and a change process of each parameter; and in addition, includes at least the measurement statistical values, the time series identifiers, and the event identifiers of each parameter. Each parameter data trend analysis area in the multi-parameter analysis report is associated with each parameter analysis page in the report attachment. In response to clicking on a certain data trend analysis area on a screen displaying the multi-parameter analysis report, the multi-parameter analysis report automatically jumps to the corresponding parameter analysis page. In this way, the medical staff can view page by page the measurement statistical values, the analysis statistical charts, the waveform data, the numerical data and the marked event data, and perform a zoom in operation, a zoom out operation, or a scrolling operation on the screen, and select a range of data for viewing.

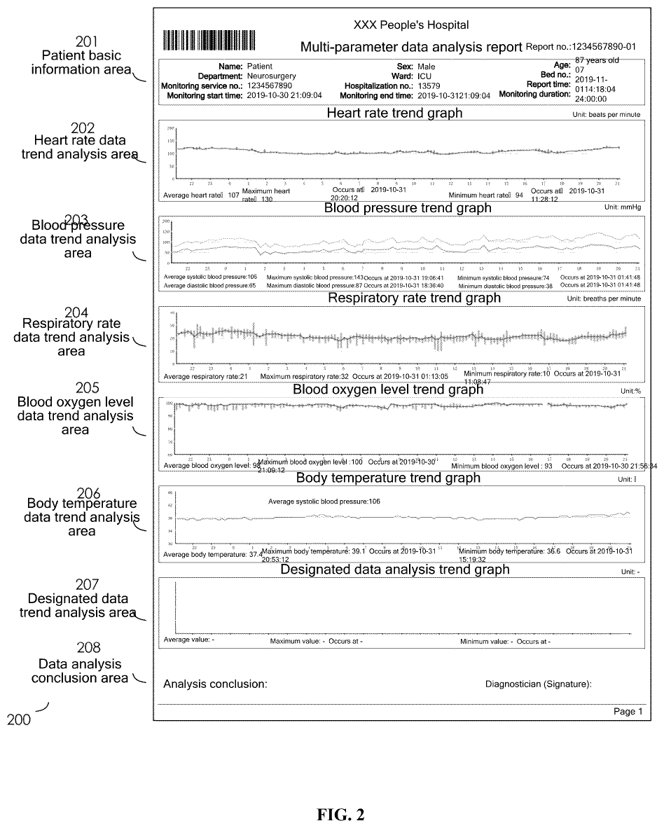

A framework of a multi-parameter analysis report 200 as shown in FIG. 2 includes a patient basic information area 201, a heart rate data trend analysis area 202, a blood pressure data trend analysis area 203, a respiratory rate data trend analysis area 204, and a blood oxygen saturation level data trend analysis area 205, a body temperature data trend analysis area 206, a designated data trend analysis area 207, and a data analysis conclusion area 208. The system software or the data analysis report generation software imports the measurement statistical values, the time series identifiers, and the event identifiers of each parameter into the multi-parameter analysis report framework, to generate the multi-parameter analysis report 200.

The patient basic information area 201 in this embodiment includes at least a name, an age, a sex, an ethnicity, a department and ward, a bed number, a hospitalization number, a disease type, and monitoring start and end time of a patient.

The heart rate data trend analysis area 202, the blood pressure data trend analysis area 203, the respiratory rate data trend analysis area 204, the blood oxygen saturation level data trend analysis area 205, the body temperature data trend analysis area 206, and the designated data trend analysis area 207 in this embodiment have an ordinate set as a numerical scale and an abscissa set as a time scale, configured for displaying a state in a unit time and a change process of each parameter; and in addition, includes at least the measurement statistical values, the time series identifiers and the event identifiers of each parameter. The heart rate data trend analysis area 202 in FIG. 2 is used as an example. The multi-parameter data to be processed is divided into several data segments of unit time. The heart rate measurement statistical values (including the average value, the maximum value, and the minimum value) of each data segment of unit time and the multi-parameter data to be processed are calculated. Then, the heart rate trend graph is drawn using the heart rate average values of the data segments in the form of the line graph, and the measurement statistical values are listed under the trend graph.

The designated data trend analysis area 207 in this embodiment is configured for displaying a trend analysis of certain parameter data that is designated by a doctor and needs intensive monitoring for patients with different diseases (such as patients with respiratory failure, and patients in shock), which includes at least a perfusion index, an end-tidal carbon dioxide, an airway flow/pressure, a central venous pressure, a pulse pressure, a stroke volume, an intracranial pressure, and a bispectral index.

The data analysis conclusion area 208 in this embodiment is configured for displaying a preliminary conclusion obtained through comprehensive analysis of the changes of various parameters, and a signature of an analysis technician or a doctor in charge of the data service.

The multi-parameter analysis report 200 in this embodiment is also provided with an attachment frame constituted by parameter analysis pages. The parameter analysis pages use analysis report templates that are commonly used in relevant medical disciplines, such as analysis report templates of electrocardiography, blood pressure, respiration, blood oxygen saturation level, body temperature, central venous pressure, invasive blood pressure, intracranial pressure, and electroencephalography (For example, the parameter analysis page of electrocardiography data uses a commonly used dynamic electrocardiography analysis report template, and the parameter analysis page of non-invasive blood pressure uses a commonly used dynamic blood pressure analysis report template). The system software or the data analysis report generation software imports the measurement statistical values, the analysis statistical charts, the waveform data, the numerical data, the time series identifiers, and the event identifiers into the analysis page templates, so as to generate the attachment of the multi-parameter analysis report. These analysis report templates are commonly used, thus are not detailed herein.

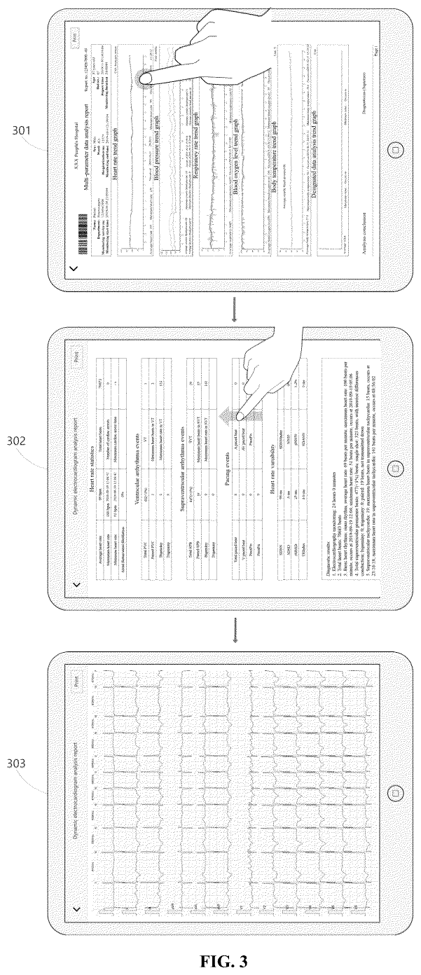

FIG. 3 shows a schematic diagram of viewing an attachment of the multi-parameter analysis report. Under the control of the medical staff terminal device, each parameter trend analysis area in the multi-parameter analysis report 200 is associated with each parameter analysis page in the attachment. As shown in FIG. 301, in response to a medical staff clicking on the heart rate trend analysis area on the screen, the current page automatically jumps to a dynamic electrocardiogram report page as shown in FIG. 302, so that the medical staff can view the measurement statistical values and abnormal events statistical values of the patent's electrocardiography data obtained during the multi-parameter monitoring process. By sliding on the screen of the terminal device, the medical staff can view page by page the statistical charts, the heart rate variability analysis chart, the waveform data as shown in FIG. 303, the numerical data, and marked event data. In addition, the medical staff can perform a zoom in operation, a zoom out operation, or a scrolling operation on the screen for viewing, and select a range of data for viewing. In response to clicking a return button on the screen by the medical staff, the page being currently displayed on the screen may automatically jump to the multi-parameter analysis report 200. This allows the medical staff to quickly understand the change processes of the patient's physiological and pathological data, and related data details, which improves medical quality and work efficiency, thereby reducing the work burden on medical staffs.

Compared with the related art, the multi-parameter analysis report 200 in this embodiment and further solutions show the change process, the event statuses and the time points of each parameter data that the medical staff pays close attention to, as well as the data analysis conclusion. Based on the data changes, the medical staff can formulate the diagnosis and treatment plan and the nursing care plan. Also, the medical staff can access patient's history reports for comparison and analysis, which exploits the value of the multi-parameter data in diagnosis and treatment. The attachment of the multi-parameter analysis report may be viewed on the medical staff terminal device, which helps the medical staff to understand more details about each parameter data. This improves medical quality and work efficiency, thereby overcoming the deficiencies of the existing multi-parameter monitoring device technology.

This embodiment provides the change trends and processes of the patient's physiological and pathological data during the multi-parameter monitoring process, which comprehensively reflects patient's medical condition and treatment effect. According to the change trend of each parameter, the medical staff can quickly determine disease progression and severity, so as to make an early detection of complications, and thereby providing further diagnosis and treatment to prevent deterioration. In addition, the medical staff gains more details about the data with the knowledge of the attachment. This embodiment improves medical quality and work efficiency, as well as reduces the work burden on the medical staff, thus having a wide range of applicability.

The multi-parameter analysis report and the report attachment generated in the present disclosure enable the medical staff to quickly and comprehensively obtain patient's physiological and pathological information, so as to provide a more targeted diagnosis and treatment plan and nursing care plan, which effectively alleviates the work burden on the medical staff. Therefore, after the multi-parameter analysis report and the report attachment are generated, the client with the first mode permission and the client with the second mode permission may also transmit the multi-parameter analysis report, the report attachment, and the unique identifier index to an associated medical staff terminal. The medical staff terminal is installed with the data management software, which is configured to perform data interaction with the client and the application server, and allow the medical staff to view the multi-parameter analysis report and the attachment. The medical staff terminal includes at least one or more of the following: a desktop computer, a large touch liquid crystal display screen, a mobile device.

Optionally, the application server also receives a report acquisition instruction transmitted by the medical staff terminal. The report acquisition instruction includes a unique identifier index of a patient or a statistical parameter. In a case that the unique identifier index is included, the application server retrieves a multi-parameter analysis report and a report attachment according to the unique identifier index, and transmits the multi-parameter analysis report and the report attachment to the medical staff terminal. In a case that the statistical parameter is included, the application server retrieves a multi-parameter analysis report and a report attachment that satisfy the statistical parameter, and transmits the multi-parameter analysis report and the report attachment to the medical staff terminal. Optionally, the application server further receives a report archiving instruction transmitted by the client or the medical staff terminal with a report archiving permission. The report archiving instruction includes the unique identifier index. The application server retrieves a multi-parameter analysis report and a report attachment according to the unique identifier index, and transmits the multi-parameter analysis report and the report attachment to a hospital information management system, an electronic medical record system, or a critical clinical information system, so as to store the multi-parameter analysis report and the report attachment in an electronic medical record of the patient.

The above solution solves the problem that the existing electronic medical records do not store the multi-parameter analysis report files. In addition, the report files in the text format effectively reduce the pressure of the huge amount of multi-parameter data on an upper-level information management system. In this embodiment, the client controls the application server to transmit the report files or a link to the medical staff terminal device for viewing. The medical staff terminal device may log in the system, to search and retrieve the data and the report files, which frees the medical staff from tedious work of manually inputting and modifying various vital sign data charts. The multi-parameter data forms a closed loop between clinical application and hospital management, which improves medical quality and work efficiency, as well as enhances the quality of the electronic medical records, thereby reducing the work burden on the medical staff. Thus, the present disclosure has a broad range of applicability.

### Embodiment 2

Another embodiment of the present disclosure provides a system for generating a data analysis report of a multi-parameter monitoring device, including: one or more clients, configured to transmit a report generation instruction to an application server, the report generation instruction including a client identifier that is configured for indicating a permission of the client; the application server, configured to receive multi-parameter data transmitted in real time by the multi-parameter monitoring device; and further configured to receive the report generation instruction transmitted by the client, in a case that the permission of the client is a first mode permission, transmit the multi-parameter data to the client, to allow the client to process the multi-parameter data, so as to generate a multi-parameter analysis report and a report attachment, and in a case that the permission of the client is a second mode permission, process the multi-parameter data to generate the multi-parameter analysis report and the report attachment, and transmit the multi-parameter analysis report and the report attachment to the client; and a database server, configured to store the multi-parameter data received by the application server.

Optionally, the system also includes a world wide web (WEB) server. The client transmits the report generation instruction to the application server through the WEB server.

Optionally, the system also includes a mobile storage apparatus. The application server receives the multi-parameter data transmitted by the multi-parameter monitoring device, including: the mobile storage apparatus connecting to an interface of the multi-parameter monitoring device, to download the multi-parameter data; and the client connecting to an interface of the mobile storage apparatus, to upload the read multi-parameter data to the application server.

Optionally, the application server is also configured to configure a unique identifier for the multi-parameter data after receiving the multi-parameter data transmitted in real time by the multi-parameter monitoring device. The database server includes a first storage area, a second storage area, and a third storage area. The first storage area of the database server is configured to store the configured unique identifier, and establish a unique identifier index based on the unique identifier. The second storage area of the database server is configured to store the multi-parameter data received by the application server, and mark the multi-parameter data according to the unique identifier of the multi-parameter data. The third storage area of the database server is configured to store the generated multi-parameter analysis report and the report attachment, and mark the generated multi-parameter analysis report and the report attachment according to the unique identifier of the multi-parameter data. The unique identifier index established by the first storage area has a mutual mapping relationship with the second storage area and the third storage area, respectively, to realize unified management of information stored in the first storage area, the second storage area, and the third storage area that is associated by the unique identifier.

Optionally, after receiving the multi-parameter data transmitted in real time by the multi-parameter monitoring device, the application server is also configured to perform the following operations: counting a time length of the received multi-parameter data; if the time length of the received multi-parameter data reaches a predetermined time length, transmitting the unique identifier index of the multi-parameter data of the predetermined time length to the client; and if the time length of the multi-parameter data is less than the predetermined time length and no longer increases, transmitting the unique identifier index of the multi-parameter data whose time length is less than the predetermined time length and no longer increases to the client. In this case, the client transmits the report generation instruction to the application server according to the received unique identifier index, so as to process the multi-parameter data to generate the multi-parameter analysis report and the report attachment.

Optionally, the report generation instruction also includes a data time period parameter for report generation. The application server is also configured to determine to-be-processed multi-parameter data of a corresponding time period length based on the data time period parameter for report generation, and generate the multi-parameter analysis report and the report attachment based on the determined multi-parameter data of the corresponding time period length.

Optionally, the multi-parameter data includes patient information, waveform data, and numerical data.

Optionally, the generation of the multi-parameter analysis report and the report attachment includes: processing the multi-parameter data, to obtain measurement statistical values, analysis statistical charts, time series identifiers, and event identifiers, the measurement statistical values including an average value, a maximum value, a minimum value, and a variability analysis value; importing the measurement statistical values, the time series identifiers, the event identifiers, and patient basic information obtained by mapping the unique identifier index into a multi-parameter analysis report framework, to generate the multi-parameter analysis report; and importing the measurement statistical values, the analysis statistical charts, the time series identifiers, the event identifiers, the waveform data, and the numerical data into a multi-parameter analysis report attachment framework, to generate the report attachment. The multi-parameter analysis report is configured for displaying a change trend of each parameter and a data analysis conclusion, and the report attachment is configured for displaying an analysis report of each parameter including the waveform data and the numerical data.

Optionally, the multi-parameter analysis report framework includes at least a patient basic information area, and further includes one or more of following parameter data trend analysis areas: a heart rate data trend analysis area, a blood pressure data trend analysis area, a respiratory rate data trend analysis area, a blood oxygen saturation level data trend analysis area, a body temperature data trend analysis area, and a designated data trend analysis area; and a data analysis conclusion area.

Optionally, the report generation instruction also includes a setting parameter of the designated data trend analysis area, according to which the application server sets content of the designated data trend analysis area; and the report generation instruction also includes a setting parameter of the data analysis conclusion area, according to which the application server sets content of the data analysis conclusion area.

Optionally, the parameter data trend analysis area has an ordinate set as a numerical scale and an abscissa set as a time scale, configured for displaying a state in a unit time and a change process of each parameter, and includes at least the measurement statistical values, the time series identifiers, and the event identifiers of each parameter.

Optionally, the designated data trend analysis area is configured for displaying a trend analysis graph of parameter data that is preset and needs intensive monitoring for patients with different diseases. The parameter data that needs intensive monitoring includes one or more of following parameters: a perfusion index, a partial pressure of end-tidal carbon dioxide, an airway flow/pressure, a central venous pressure, a pulse pressure, a stroke volume, an intracranial pressure, and a bispectral index.

Optionally, each of the parameter data trend analysis areas in the multi-parameter analysis report is associated with each parameter analysis page in the report attachment. In response to clicking on a certain data trend analysis area on a screen displaying the multi-parameter analysis report, the multi-parameter analysis report automatically jumps to a corresponding parameter analysis page, to allow a user to view page by page the measurement statistical values, the analysis statistical charts, the waveform data, the numerical data, and marked event data, and perform a zoom in operation, a zoom out operation, or a scrolling operation on the screen for viewing, and select a range of data for viewing.

Optionally, after the multi-parameter analysis report and the report attachment are generated, the client with the first mode permission and the client with the second mode permission are also configured to transmit the multi-parameter analysis report, the report attachment, and the unique identifier index to an associated medical staff terminal.

Optionally, the system also includes a medical staff terminal, configured to transmit a report acquisition instruction to the application server. The report acquisition instruction includes a unique identifier index of a patient or a statistical parameter.

The application server is also configured to receive the report acquisition instruction transmitted by the medical staff terminal When including the unique identifier index, the application server retrieves a multi-parameter analysis report and a report attachment according to the unique identifier index, and transmits the multi-parameter analysis report and the report attachment to the medical staff terminal; and

when including the statistical parameter, the application server retrieves a multi-parameter analysis report and a report attachment that satisfy the statistical parameter, and transmits the multi-parameter analysis report and the report attachment to the medical staff terminal.

Optionally, the application server is also configured to receive a report archiving instruction transmitted by a client or a medical staff terminal with a report archiving permission. The report archiving instruction includes the unique identifier index. The application server retrieves a multi-parameter analysis report and a report attachment according to the unique identifier index, and transmits the multi-parameter analysis report and the report attachment to a hospital information management system, an electronic medical record system, or a critical clinical information system, to store the multi-parameter analysis report and the report attachment in a patient electronic medical record.

A specific implementation process of the device embodiment may refer to the foregoing method embodiment, which is not detailed herein. Since this embodiment has the same principle as the foregoing method embodiment, the system has the corresponding technical effects of the foregoing method embodiment.

### Embodiment 3

This embodiment describes an actual application process of the foregoing method embodiment and system embodiment.

According to different application scenarios and requirements of users, devices such as a client, an application server, a database server, a medical staff terminal, and a printer, as well as a system management software, a data analysis report generation software, a mobile service software, a data management software may be combined to form a client/server (C/S) architecture, a browser/server (B/S) architecture, or a local client architecture, etc., so that a huge amount of multi-parameter data from various departments of a hospital can be centrally processed to generate a multi-parameter analysis report.

The application server is installed with the system management software, configured to control system operation. The medical staff terminal is installed with the data management software, configured to receive report files and perform data interaction. The desktop computer is installed with the data analysis report generation software or a browser. The mobile device is installed with the mobile service software or a browser, configured to support the client device to operate in systems under different architectures. The mobile device includes at least a tablet computer, a mobile computing device, a handheld mobile terminal, and a smartphone.

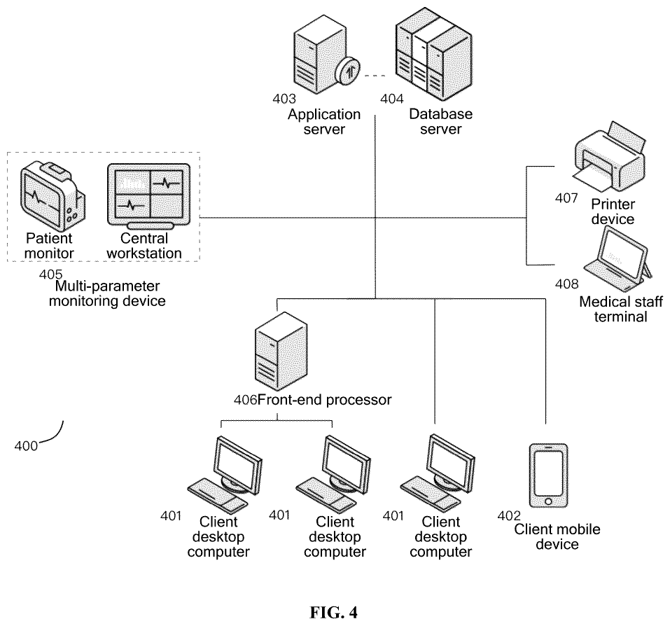

As shown in FIG. 4, a system 400 for generating a data analysis report of a multi-parameter monitoring device uses the C/S architecture, including: a client desktop computer 401 (the desktop computer is installed with the data analysis report generation software, and has the first mode permission), a client mobile device 402 (the mobile device is installed with the mobile service software that can transmit an instruction to the application server, and has the second mode permission), an application server 403, a database server 404, a multi-parameter monitoring device 405, a front-end processor 406, a printer device 407, and a medical staff terminal device 408. The front-end processor is configured to buffer the multi-parameter data. The printer device is configured to print the generated multi-parameter analysis report and report attachment. The functions of remaining devices refer to the foregoing embodiments.

In the C/S architecture, each device has a following interaction process.

Step 1. The application server receives the multi-parameter data transmitted in real time by the multi-parameter monitoring device, the multi-parameter data including patient information, waveform data, and numerical data; and stores the received multi-parameter data in the database server.

Specifically, the received multi-parameter data is stored in a second storage area (namely a data storage area) of the database server. In addition, a unique identifier of the multi-parameter data is configured and stored in a first storage area (namely an index storage area) of the database server, and a unique identifier index is established based on the unique identifier.

Step 2. The application server receives a report generation instruction transmitted by the client. The instruction includes a client identifier, which is configured for indicating a permission of the client. In this embodiment, when the client identifier indicates that the client is the desktop computer, it represents that the client has the first mode permission; and when the client identifier indicates that the client is the client mobile device, it represents that the client has the second mode permission. The client device identifier is configured for distinguishing the client is the desktop computer or the mobile device.

Step 3. The application server determines whether it has stored the client device identifier. If the client device identifier has not been stored, the application server transmits feedback information that the client does not have the report generation function to the client; and otherwise, the application server performs the following operations.

If the client device identifier is the desktop computer, the application server transmits the multi-parameter data to the client, and the desktop computer downloads the multi-parameter data to a local storage for calculation processing. The measurement statistical values, the time series identifiers, the event identifiers, and the patient basic information obtained by mapping the unique identifier index are imported in the multi-parameter analysis report framework, to generate the multi-parameter analysis report. The measurement statistical values, the analysis statistical charts, the time series identifiers, the event identifiers, as well as the multi-parameter waveform data and the numerical data are imported in the multi-parameter analysis report attachment framework, to generate the multi-parameter analysis report attachment. In addition, the client desktop computer marks the multi-parameter analysis report and the attachment according to the unique identifier of the data, and stores the multi-parameter analysis report and the attachment in a report file storage area (namely the third storage area) of the database server.

If the client device identifier is the mobile device, the application server processes the multi-parameter data, to generate the multi-parameter analysis report and the attachment in the above manner. The application server marks the multi-parameter analysis report and the attachment according to the unique identifier of the data, and stores the multi-parameter analysis report and the attachment in the report file storage area of the database server.

In addition, if the client device identifier is the desktop computer, the client desktop computer also controls the application server to transmit the report files to the medical staff terminal device for viewing, and at the same time, controls the printer to output the multi-parameter analysis report and the attachment.

If the client device identifier is the mobile device, the client mobile device transmits an instruction to the application server, to control the application server to transmit the report files to the medical staff terminal device for viewing. The client mobile device downloads the report files to a memory, and controls the printer to output the multi-parameter analysis report and the attachment.

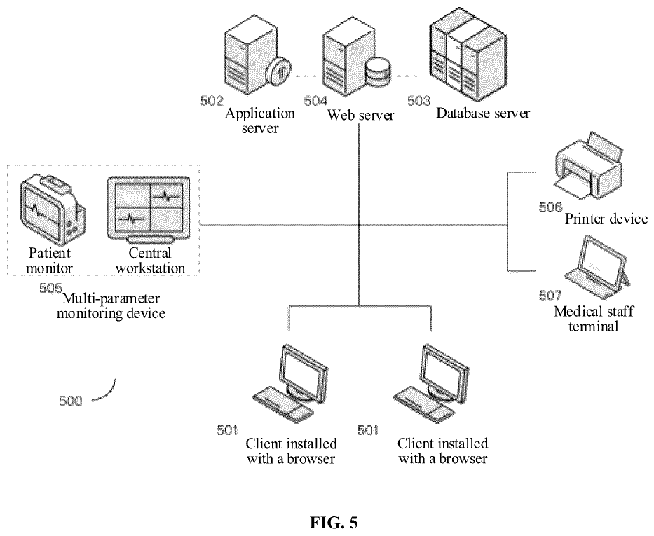

As shown in FIG. 5, a system 500 for generating a data analysis report of a multi-parameter monitoring device uses the B/S architecture, including: a client device 501 installed with a browser, an application server 502, a database server 503, a WEB server 504, a multi-parameter monitoring device 505, a printer device 506, and a medical staff terminal device 507.

In the B/S architecture, each device has a following interaction process.

Step S1. This step is the same as above, which is not detailed herein.

Step S2. The application server receives a report generation instruction that is transmitted by the client browser and successfully authenticated by the WEB server. The report generation instruction includes a client browser identifier, and the permission of the client indicated by the client browser identifier is the second mode permission.

Step S3. In response to determining that the permission of the client is the second mode permission, the application server processes the multi-parameter data, to generate the multi-parameter analysis report and the attachment in the above manner. The application server marks the multi-parameter analysis report and the attachment according to the unique identifier of the data, and stores the multi-parameter analysis report and the attachment in the report file storage area of the database server.

In addition, after the multi-parameter analysis report and the attachment are generated, the client browser also transmits an instruction to the WEB server, to control the application server to transmit the report files to the medical staff terminal device for viewing. The client browser also downloads the report files to a memory, and controls the printer to output the multi-parameter analysis report and the attachment.

It should be noted that due to the characteristics of the B/S architecture, transmission and interaction of uplink and downlink data information between the client browser and the application server need to be performed by the WEB server.

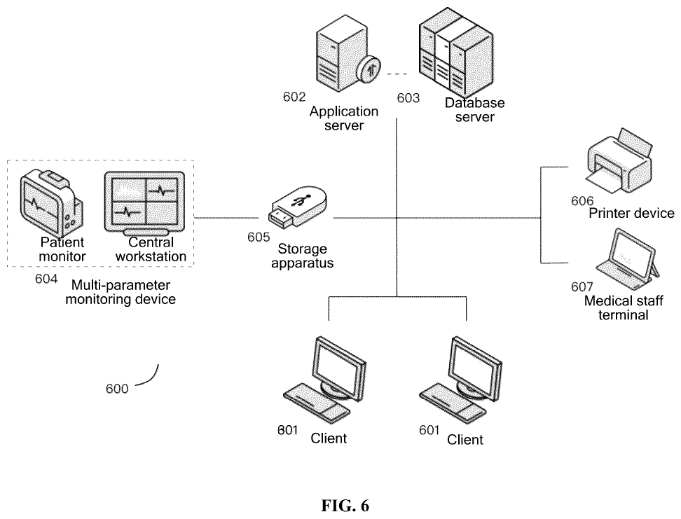

As shown in FIG. 6, a system 600 for generating a data analysis report of a multi-parameter monitoring device uses the local client architecture, including: a client device 601, an application server 602, a database server 603, a multi-parameter monitoring device 604, a mobile storage apparatus 605, a printer device 606, and a medical staff terminal device 607.

In this system, the mobile storage apparatus is connected to an interface of the multi-parameter monitoring device, to download the multi-parameter data. The client is connected to an interface of the mobile storage apparatus, to read the multi-parameter data, and upload the multi-parameter data to the application server. The subsequent processing process may refer to the foregoing method.

In addition, when the client device fails to connect to the application server, the report files may be generated in the following way:

The client device stores the multi-parameter data in a memory of the client device, after reading the multi-parameter data from the interface of the mobile storage apparatus.

The client device processes the multi-parameter data, to generate the multi-parameter analysis report and the attachment, and stores the multi-parameter analysis report and the attachment in a memory of the client device.

In this case, the client device is installed with a software program configured for generating the multi-parameter analysis report and the attachment.

From above, the multi-parameter analysis report and the attachment can be generated in different system architectures, which greatly expands application scenarios of the present disclosure, thereby satisfying various application scenarios and requirements of hospitals.

Those skilled in the art can understand that relevant hardwares can be instructed through computer programs to implement all or part of processes in the method according to the above examples, and the programs can be stored in a computer-readable storage medium. The computer-readable storage medium may be a magnetic disk, an optical disk, a read-only memory (ROM), a random-access memory (RAM), or the like.

The above merely describes preferred specific implementations of the present application, but a protection scope of the present application is not limited thereto. Any person skilled in the art can easily conceive modifications or replacements within the technical scope of the present application, and these modifications or replacements shall fall within the protection scope of the present application.

1-32. (canceled)

33. A method for generating a data analysis report of a multi-parameter monitoring device, comprising:
receiving, by an application server, multi-parameter data transmitted in real time by the multi-parameter monitoring device, and storing the received multi-parameter data in a database server;
receiving, by the application server, a report generation instruction transmitted by a client, the instruction comprising a client identifier that is configured for indicating a permission of the client; and
in a case that the permission of the client is a first mode permission, transmitting, by the application server, the multi-parameter data to the client, to allow the client to process the multi-parameter data, so as to generate a multi-parameter analysis report and a report attachment; and
in a case that the permission of the client is a second mode permission, processing, by the application server, the multi-parameter data to generate the multi-parameter analysis report and the report attachment, and transmitting the multi-parameter analysis report and the report attachment to the client.

34. The method for generating a data analysis report of a multi-parameter monitoring device according to claim 33, wherein after the receiving, by an application server, multi-parameter data transmitted in real time by the multi-parameter monitoring device, the method further comprises:
configuring a unique identifier for the multi-parameter data;
storing the configured unique identifier in a first storage area of the database server, and establishing a unique identifier index based on the unique identifier;
storing the received multi-parameter data in a second storage area of the database server, and marking the multi-parameter data according to the unique identifier of the multi-parameter data; and
storing the generated multi-parameter analysis report and the report attachment in a third storage area of the database server, and marking the generated multi-parameter analysis report and the report attachment according to the unique identifier of the multi-parameter data;
wherein the unique identifier index established by the first storage area has a mutual mapping relationship with the second storage area and the third storage area, respectively, to provide unified management of information stored in the first storage area, the second storage area, and the third storage area that is associated by the unique identifier.

35. The method for generating a data analysis report of a multi-parameter monitoring device according to claim 34, wherein after the receiving, by an application server, multi-parameter data transmitted in real time by the multi-parameter monitoring device, the method further comprises:
counting, by the application server, a time length of the received multi-parameter data;
in a case that the time length of the multi-parameter data reaches a predetermined time length, transmitting the unique identifier index of the multi-parameter data of the predetermined time length to the client
in a case that the time length of the multi-parameter data is less than the predetermined time length and no longer increases, transmitting the unique identifier index of the multi-parameter data whose time length is less than the predetermined time length and no longer increases to the client; and
transmitting, by the client, the report generation instruction to the application server according to the received unique identifier index,
wherein the report generation instruction further comprises a data time period parameter for report generation;
determining, by the application server, to-be-processed multi-parameter data of a corresponding time period length based on the data time period parameter for report generation, and generating the multi-parameter analysis report and the report attachment based on the determined multi-parameter data of the corresponding time period length.

36. The method for generating a data analysis report of a multi-parameter monitoring device according to claim 34, wherein the multi-parameter data comprises patient information, waveform data, and numerical data;
wherein the generating a multi-parameter analysis report and a report attachment comprises:
processing the multi-parameter data, to obtain measurement statistical values, analysis statistical charts, time series identifiers, and event identifiers, the measurement statistical values comprising an average value, a maximum value, a minimum value, and a variability analysis value;
importing the measurement statistical values, the time series identifiers, the event identifiers, and patient basic information obtained by mapping the unique identifier index into a multi-parameter analysis report framework, to generate the multi-parameter analysis report; and
importing the measurement statistical values, the analysis statistical charts, the time series identifiers, the event identifiers, the waveform data, and the numerical data into a multi-parameter analysis report attachment framework, to generate the report attachment
wherein the multi-parameter analysis report is configured for displaying a change trend of each parameter and a data analysis conclusion, and the report attachment is configured for displaying an analysis report of each parameter comprising the waveform data and the numerical data;
the multi-parameter analysis report framework comprises at least a patient basic information area;
and further comprises:
one or more of following parameter data trend analysis areas: a heart rate data trend analysis area, a blood pressure data trend analysis area, a respiratory rate data trend analysis area, a blood oxygen saturation level data trend analysis area, a body temperature data trend analysis area, and a designated data trend analysis area; and
a data analysis conclusion area.

37. The method for generating a data analysis report of a multi-parameter monitoring device according to claim 36, wherein,
the report generation instruction further comprises a setting parameter of the designated data trend analysis area, according to which the application server sets content of the designated data trend analysis area; and
the report generation instruction further comprises a setting parameter of the data analysis conclusion area, according to which the application server sets content of the data analysis conclusion area;
wherein each of the parameter data trend analysis areas comprises an ordinate set as a numerical scale and an abscissa set as a time scale, configured for displaying a state in a unit time and a change process of each parameter, and comprises at least the measurement statistical values, the time series identifiers, and the event identifiers of each parameter;
wherein the designated data trend analysis area is configured for displaying a trend analysis graph of parameter data that is preset and needs intensive monitoring for patients with different diseases;
wherein the parameter data that needs intensive monitoring comprises one or more of following parameters: a perfusion index, a partial pressure of end-tidal carbon dioxide, an airway flow/pressure, a central venous pressure, a pulse pressure, a stroke volume, an intracranial pressure, and a bispectral index.

38. The method for generating a data analysis report of a multi-parameter monitoring device according to claim 37, wherein,
each of the parameter data trend analysis areas in the multi-parameter analysis report is associated with each parameter analysis page in the report attachment, and in response to clicking on a certain data trend analysis area on a screen displaying the multi-parameter analysis report, the multi-parameter analysis report automatically jumps to a corresponding parameter analysis page, to allow a user to view page by page the measurement statistical values, the analysis statistical charts, the waveform data, the numerical data, and marked event data, and perform a zoom in operation, a zoom out operation, or a scrolling operation on the screen for viewing, and select a range of data for viewing.

39. The method for generating a data analysis report of a multi-parameter monitoring device according to claim 34, wherein,
the client with the first mode permission and the client with the second mode permission transmit the multi-parameter analysis report, the report attachment, and the unique identifier index to an associated medical staff terminal, after the multi-parameter analysis report and the report attachment are generated;
wherein the method further comprises:
receiving, by the application server, a report acquisition instruction transmitted by the medical staff terminal, the report acquisition instruction comprising a unique identifier index of a patient or a statistical parameter;
in a case that the unique identifier index is comprised, retrieving, by the application server, a multi-parameter analysis report and a report attachment according to the unique identifier index, and transmitting the multi-parameter analysis report and the report attachment to the medical staff terminal; and
in a case that the statistical parameter is comprised, retrieving, by the application server, a multi-parameter analysis report and a report attachment that satisfy the statistical parameter, and transmitting the multi-parameter analysis report and the report attachment to the medical staff terminal.

40. The method for generating a data analysis report of a multi-parameter monitoring device according to claim 39, wherein the method further comprises:
receiving, by the application server, a report archiving instruction transmitted by a client or a medical staff terminal with a report archiving permission, the report archiving instruction comprising the unique identifier index; and retrieving, by the application server, a multi-parameter analysis report and a report attachment according to the unique identifier index, and transmitting the multi-parameter analysis report and the report attachment to a hospital information management system, an electronic medical record system, or a critical clinical information system, to store the multi-parameter analysis report and the report attachment in a patient electronic medical record.

41. The method for generating a data analysis report of a multi-parameter monitoring device according to claim 33, wherein the receiving, by the application server, a report generation instruction transmitted by a client further comprises:
receiving, by the application server, the report generation instruction that is transmitted by a client browser and successfully authenticated by a world wide web (WEB) server, the report generation instruction comprising a client browser identifier, and the permission of the client indicated by the client browser identifier being the second mode permission.

42. The method for generating a data analysis report of a multi-parameter monitoring device according to claim 33, wherein the receiving, by an application server, multi-parameter data transmitted in real time by the multi-parameter monitoring device comprises:
connecting, by a mobile storage apparatus, to an interface of the multi-parameter monitoring device, to download the multi-parameter data; and
connecting, by the client, to an interface of the mobile storage apparatus, to upload read multi-parameter data to the application server.

43. A system for generating a data analysis report of a multi-parameter monitoring device, comprising:
one or more clients, configured to transmit a report generation instruction to an application server, the report generation instruction comprising a client identifier that is configured for indicating a permission of the client
the application server, configured to receive multi-parameter data transmitted in real time by the multi-parameter monitoring device; and further configured to receive the report generation instruction transmitted by the client, in a case that the permission of the client is a first mode permission, transmit the multi-parameter data to the client, to allow the client to process the multi-parameter data, so as to generate a multi-parameter analysis report and a report attachment, and in a case that the permission of the client is a second mode permission, process the multi-parameter data to generate the multi-parameter analysis report and the report attachment, and transmit the multi-parameter analysis report and the report attachment to the client; and
a database server, configured to store the multi-parameter data received by the application server.

44. The system for generating a data analysis report of a multi-parameter monitoring device according to claim 43, wherein,
the application server is further configured to configure a unique identifier for the multi-parameter data after receiving the multi-parameter data transmitted in real time by the multi-parameter monitoring device;
the database server comprises a first storage area, a second storage area, and a third storage area; wherein,
the first storage area of the database server is configured to store the configured unique identifier, and establish a unique identifier index based on the unique identifier;
the second storage area of the database server is configured to store the multi-parameter data received by the application server, and mark the multi-parameter data according to the unique identifier of the multi-parameter data; and
the third storage area of the database server is configured to store the generated multi-parameter analysis report and the report attachment, and mark the generated multi-parameter analysis report and the report attachment according to the unique identifier of the multi-parameter data;
wherein the unique identifier index established by the first storage area has a mutual mapping relationship with the second storage area and the third storage area, respectively, to provide unified management of information stored in the first storage area, the second storage area, and the third storage area that is associated by the unique identifier.

45. The system for generating a data analysis report of a multi-parameter monitoring device according to claim 44, wherein,
the application server is further configured to count a time length of the received multi-parameter data, after receiving the multi-parameter data transmitted in real time by the multi-parameter monitoring device;
in a case that the time length of the received multi-parameter data reaches a predetermined time length, transmit the unique identifier index of the multi-parameter data of the predetermined time length to the client; and
in a case that the time length of the multi-parameter data is less than the predetermined time length and no longer increases, transmit the unique identifier index of the multi-parameter data whose time length is less than the predetermined time length and no longer increases to the client and the client is further configured to transmit the report generation instruction to the application server according to the received unique identifier index;
wherein the report generation instruction further comprises a data time period parameter for report generation; and
the application server is further configured to determine to-be-processed multi-parameter data of a corresponding time period length based on the data time period parameter for report generation, and generate the multi-parameter analysis report and the report attachment based on the determined multi-parameter data of the corresponding time period length.

46. The system for generating a data analysis report of a multi-parameter monitoring device according to claim 44, wherein the multi-parameter data comprises patient information, waveform data, and numerical data;
wherein the generation of the multi-parameter analysis report and the report attachment comprises:
processing the multi-parameter data, to obtain measurement statistical values, analysis statistical charts, time series identifiers, and event identifiers, the measurement statistical values comprising an average value, a maximum value, a minimum value, and a variability analysis value;
importing the measurement statistical values, the time series identifiers, the event identifiers, and patient basic information obtained by mapping the unique identifier index into a multi-parameter analysis report framework, to generate the multi-parameter analysis report; and
importing the measurement statistical values, the analysis statistical charts, the time series identifiers, the event identifiers, the waveform data, and the numerical data into a multi-parameter analysis report attachment framework, to generate the report attachment;
wherein the multi-parameter analysis report is configured for displaying a change trend of each parameter and a data analysis conclusion, and the report attachment is configured for displaying an analysis report of each parameter comprising the waveform data and the numerical data;
the multi-parameter analysis report framework comprises at least a patient basic information area;
and further comprises:
one or more of following parameter data trend analysis areas: a heart rate data trend analysis area, a blood pressure data trend analysis area, a respiratory rate data trend analysis area, a blood oxygen saturation level data trend analysis area, a body temperature data trend analysis area, and a designated data trend analysis area; and
a data analysis conclusion area.

47. The system for generating a data analysis report of a multi-parameter monitoring device according to claim 46, wherein,
the report generation instruction further comprises a setting parameter of the designated data trend analysis area, according to which the application server sets content of the designated data trend analysis area; and
the report generation instruction further comprises a setting parameter of the data analysis conclusion area, according to which the application server sets content of the data analysis conclusion area;
wherein each of the parameter data trend analysis areas comprises an ordinate set as a numerical scale and an abscissa set as a time scale, configured for displaying a state in a unit time and a change process of each parameter, and comprises at least the measurement statistical values, the time series identifiers, and the event identifiers of each parameter;
wherein the designated data trend analysis area is configured for displaying a trend analysis graph of parameter data that is preset and needs intensive monitoring for patients with different diseases;
wherein the parameter data that needs intensive monitoring comprises one or more of following parameters: a perfusion index, a partial pressure of end-tidal carbon dioxide, an airway flow/pressure, a central venous pressure, a pulse pressure, a stroke volume, an intracranial pressure, and a bispectral index.

48. The system for generating a data analysis report of a multi-parameter monitoring device according to claim 47, wherein,
each of the parameter data trend analysis areas in the multi-parameter analysis report is associated with each parameter analysis page in the report attachment, and in response to clicking on a certain data trend analysis area on a screen displaying the multi-parameter analysis report, the multi-parameter analysis report automatically jumps to a corresponding parameter analysis page, to allow a user to view page by page the measurement statistical values, the analysis statistical charts, the waveform data, the numerical data, and marked event data, and perform a zoom in operation, a zoom out operation, or a scrolling operation on the screen for viewing, and select a range of data for viewing.

49. The system for generating a data analysis report of a multi-parameter monitoring device according to claim 44, wherein,
the client with the first mode permission and the client with the second mode permission are further configured to transmit the multi-parameter analysis report, the report attachment, and the unique identifier index to an associated medical staff terminal, after the multi-parameter analysis report and the report attachment are generated; the system further comprises a medical staff terminal, configured to transmit a report acquisition instruction to the application server, the report acquisition instruction comprising a unique identifier index of a patient or a statistical parameter; and
the application server is further configured to receive the report acquisition instruction transmitted by the medical staff terminal, in a case that the unique identifier index is comprised, retrieve a multi-parameter analysis report and a report attachment according to the unique identifier index, and transmit the multi-parameter analysis report and the report attachment to the medical staff terminal; and
in a case that the statistical parameter is comprised, retrieve a multi-parameter analysis report and a report attachment that satisfy the statistical parameter, and transmit the multi-parameter analysis report and the report attachment to the medical staff terminal.

50. The system for generating a data analysis report of a multi-parameter monitoring device according to claim 49, wherein,
the application server is further configured to receive a report archiving instruction transmitted by a client or a medical staff terminal with a report archiving permission, the report archiving instruction comprising the unique identifier index, retrieve a multi-parameter analysis report and a report attachment according to the unique identifier index, and transmit the multi-parameter analysis report and the report attachment to a hospital information management system, an electronic medical record system, or a critical clinical information system, to store the multi-parameter analysis report and the report attachment in a patient electronic medical record.

51. The system for generating a data analysis report of a multi-parameter monitoring device according to claim 43, wherein the system further comprises a WEB server; and
the application server is configured to receive the report generation instruction that is transmitted by a client browser and successfully authenticated by the WEB server, the report generation instruction comprising a client browser identifier, and the permission of the client indicated by the client browser identifier being the second mode permission.

52. The system for generating a data analysis report of a multi-parameter monitoring device according to claim 43, wherein the system further comprises a mobile storage apparatus;
the application server is configured to receive the multi-parameter data transmitted by the multi-parameter monitoring device, comprising:
a mobile storage apparatus connecting to an interface of the multi-parameter monitoring device, to download the multi-parameter data; and
the client connecting to an interface of the mobile storage apparatus, to upload read multi-parameter data to the application server.
